(19) United States
(12) Patent Application Publication
(10) Pub. No.: US 2024/0042442 A1
(43) Pub. Date: Feb. 8, 2024

(54) MICROFLUIDIC METHODS AND SYSTEMS

(71) Applicant: HIFIBIO SAS, Paris (FR)

(72) Inventors: Sami Ellouze, Paris (FR); Bingqing Shen, Paris (FR); Arnaud Reitz, Paris (FR); Annabelle Patricia Veronique Gérard, Paris (FR); Karolis Simutis, Paris (FR); Andrew Griffiths, Paris (FR); Marcel Reichen, Paris (FR)

(21) Appl. No.: 18/268,810

(22) PCT Filed: Dec. 17, 2021
(86) PCT No.: PCT/EP2021/086668

§ 371 Date: Jun. 21, 2023

(30) Foreign Application Priority Data
Dec. 22, 2020 (EP) 20306661.8

(51) Int. Cl. B01L 3/00; C12Q 1/6837; C12N 15/10

(52) U.S. Cl. CPC B01L 3/502761; C12Q 1/6837; B01L 3/502707; C12N 15/1075; B01L 2200/027; B01L 2200/0668; B01L 2200/10; B01L 2300/021; B01L 2300/047; B01L 2300/0636; B01L 2300/0867

(57) ABSTRACT

The invention relates to a microfluidic system comprising: a) a solid support comprising at least a first group of oligonucleotides, i. wherein each oligonucleotide in said group comprises a nucleic acid sequence of a first type, of a second type and/or a further type, ii. wherein said nucleic acid sequence of a first type is a barcode sequence, iii. and oligonucleotides comprising the same barcode sequence are grouped together in a group of oligonucleotides on said solid support, iv. wherein the first and further oligonucleotide groups are spatially separated on said solid support, b) wherein said one or more groups of oligonucleotide groups on said solid support are within separate reservoirs of the microfluidics system, c) wherein the one or more reservoirs are accessible to fluids, cells, chemicals and/or microdroplet by means of channels, and d) wherein each reservoir comprises comprising a group of oligonucleotides on said solid support is also trap for a microfluidic droplet.

### FIELD OF THE INVENTION

The present invention is in the field of molecular biology and relates to methods for assigning a phenotype to a genotype using droplets in microfluidic devices. The invention is also in the field of microfluidics and encompasses microfluidic devices, method for producing the same and use thereof for carrying out biological assays.

### BACKGROUND

Recent progress in single cell analysis methods, for example single cell RNA-seq methods developed by Klein (Klein et al. 2015, Cell 161(5):1187-1201) and Macosko (Macosko et al. 2015, Cell 161(5):1202-1214) or single cell epigenetics ChIP-seq methods conceived by Rotem (Rotem et al. 2015, Nat. Biotechnol. 33(11):1165-1172), enable the dissection of cell populations with higher throughput than corresponding bulk methods (Jaitin et al. 2014, Sciences 343(6172):776-779). However, sequencing data allow only an end-point measurement of a cell or cellular system and there is a growing need to include kinetics data or information around the phenotype of a cell to be included to complement and augment the genetic information obtained.

The underlying methods of functional assays are well established in bulk and have been adapted for single cell analysis methods by Agresti (Agresti et al. 2010, PNAS 107(9):4004-4009). Droplet microfluidics offers a panel of methods which can address multiple challenges such as high throughput screening using elements like single cell encapsulation, droplet sorting, droplet fusion to build phenotypic assays. For example, Mazutis describes a method for selection of droplets containing B cells producing antibodies against a target of interest using a magnetic bead to capture immunoglobulins (Mazutis et al. 2013, Nat. Prot. 8:870-891). A variation of said method is published by Eyer, where the single magnetic bead is replaced by multiple magnetic nanoparticles ensuring every cell becomes amenable to analysis (Eyer et al. 2017, Nat. Biotechnol. 35(10):977-982). These two examples demonstrate in a high-throughput fashion binding events of antibodies in droplets.

In an ideal screening system for drug discovery, selection of phenotypes of interest is not a single step process but consists of a step-wise selection of phenotypes based on a combination of different phenotypic assays, typically on binding and/or a functional read-out either in an end-point measurement or kinetically.

A key step in every phenotypic screening is the selection of reporter systems (e.g., antibodies, chemical dyes or genetically encoded fluorescent tags). In fluorescent microscopy, only a relatively small number of reporter systems can be monitored simultaneously in each cell. Multiplexing reporter systems and/or performing additional replicate experiments can increase the number of readouts used to probe cellular responses and provide useful information. However, increasing the number of reporter systems can lead to increased costs and time for screening.

In addition, to inform in a first step for phenotypic functions of cell-cell interaction/recognition and/or compound function at high throughput, and in the second step for genotyping at the single cell level, there are needs to couple in an informed way both the phenotype and genotype at the single cell level.

Microfluidics have emerged as a powerful technology for performing a diverse range of biological and chemical assays in a high-throughput manner. This technology allows high-throughput analysis of a complex sample by partitioning a bulk solution into many isolated pico- to nanoliter-sized compartments or microreactors.

However, by using methods known in the art, post-analysis retrieval of individual samples is difficult to achieve. Furthermore, mixing of reagents in these devices either requires complex architecture or is often done in bulk before compartmentalization, which may prevent initial reaction products from co-localizing with their initiating target.

Indeed, microfluidic methods for combination of phenotypic screenings with genotyping at the single cell level lack accuracy in discriminating droplets. In particular, methods for screening cells having a phenotype of interest, combined optionally with functional readout, and recovery of specific cell genotype information are highly desirable since the recovery of single cell specific genotype together with single cell specific phenotype is very challenging.

The method disclosed herein is intended to solve the above issues affecting the microfluidic methods known in the art.

The inventors have developed a microfluidic device for carrying out the method disclosed herein, wherein single cell droplets are captured in individual compartments. The single cell droplets are then selectively fused with other droplets coupling phenotype information (protein expression level, cellular pathway activation/activity, ion channel/GPCR activities) with genotypic or epigenetic information, thus allowing determining the genotype of a single cell having a phenotype of interest.

### SUMMARY OF THE INVENTION

The invention relates to a microfluidic system comprising:
a) a solid support comprising at least a first group of oligonucleotides,
   i. wherein each oligonucleotide in said group comprises a nucleic acid sequence of a first type, of a second type and/or a further type,
   ii. wherein said nucleic acid sequence of a first type is a barcode sequence
   iii. and oligonucleotides comprising the same barcode sequence are grouped together in a group of oligonucleotides on said solid support,
   iv. wherein the first and further oligonucleotide groups are spatially separated on said solid support,
b) wherein said one or more groups of oligonucleotide groups on said solid support are within separate reservoirs of the microfluidics system,
c) wherein the one or more reservoirs are accessible to fluids, cells, chemicals and/or microdroplet by means of channels, and
d) wherein each reservoir comprises comprising a group of oligonucleotides on said solid support is also trap for a microfluidic droplet.

The invention also relates to a method of attaching an oligonucleotide to a cell, the method comprising:
a) providing a microfluidic system according to the invention,
b) encapsulating a first cell in a first droplet,
c) trapping said cell in said reservoir,
d) merging a second droplet comprising a lysis composition with said first droplet, thereby allowing an oligonucleotide of said solid support to attach a nucleic acid in said cell.

The invention further relates to a method for determining a phenotype and/or a genotype of a single cell, the method comprising:
a) providing a microfluidics device comprising at least one microfluidic channel, at least a collector system comprising a plurality of reservoirs,
b) encapsulating at least one cell of a plurality of cells of a first type separately into a droplet of a first type,
optionally co-encapsulating a cell of a second type from a plurality of second type cells into each of the droplets of a first type,
c) flowing a plurality of droplets of a first type in a microfluidic channel of the microfluidics device and trapping inside each reservoir of the microfluidics device a droplet of a first type, optionally analyzing a phenotype within the droplet comprised within the reservoir,
d) flowing a plurality of droplets of a second type in a microfluidic channel and trapping inside each reservoir a second droplet of a second type,
e) merging the droplets of a first type with the droplet of a second type inside the reservoir,
f) performing at least one reaction inside the merged droplet obtained in e) and determining a readout of the reaction.

The invention further relates to a method of producing a system according to the invention. The invention also relates to a kit comprising the microfluidic system of the invention and optionally instructions for performing the method of the invention.

### BRIEF DESCRIPTION OF THE DRAWINGS

FIG. 1 shows a 3D view of the device of the invention containing the first cell droplet trapped in a reservoir, the second reagent droplet touches the first cell droplet and is localized underneath the array of barcoded oligos. Said reservoirs are organized in a way that first droplets do not touch each other, and second and further droplets do not touch each other so that fusion can only occur between the two first and second droplets trapped in the reservoir and wetting to the local spatially arranged oligos. The barcodes are organized so that they are in contact with a single second droplet.

FIG. 2 2D view of the two devices presenting two different features. The array part is designed with oligo (2) regularly spotted on slide surface (1). As second device called fluidics device (3) is designed for organizing the droplets introduced in the fluidic system. This device is also used for manipulating droplets of different types.

FIG. 3 shows a 2D view of both assembled devices described in FIG. 2. Both are then combined zo for organizing droplets in accordance with the spotted oligos on top of the slide surface. The oligos are used to react specifically with any type of material introduced into the droplet, typically a cell or a cell lysate.

FIG. 4 Fabrication process of the full assembled devices. Both devices described at FIG. 2 are separately produced. (1) The array slide is ordered from subcontractor preparing the different oligo spot at the slice surface. The oligo composition could be adapted to any type of reaction performed in droplet. For the fluidics part, the fabrication starts with (19) the production of a SU8 mold. An initial device is drawn using any type of 3D software, typically AUTOCAD. The mask is then printed and will allow a photo activation of the SU8 (resin) following the negative part of the printed mask. The excess of resin is then removed using organic solvent. An SU8 mold (19) containing the same design but in 3D will represent the positive footprint. This step is performed multiple time to create multiple layer of SU8 resin with different design. This is used for generating different features in the fluidic device and creating various type of droplet organization or manipulation. a) on the SU8 mold (19) non polymerized PDMS is casted and embarrassed the SU8 mold shape. After baking the PDMS becomes rigid and the SU8 shape are replicated as negative in the PDMS piece. b) The PDMS is removed from the SU8 mold and constitute a PDMS mold (20). c) on top of PDMS mold, a COC polymer is hot embossed on the PDMS surface. The plastic embrace the PDMS surface and replicate the negative design at the COC surface. d) After detaching the COC piece from the PDMS mold, the COC piece become the fluidics device with the known fluidic properties. e) The both array part and the COC fluidic piece are then assembled using any type of sealing (thermo sealing, double tape, glue, resin . . . ).

FIG. 5 The array slide (1) spotted with oligos is in the present example composed by three sequence type. (8) correspond to a sequence of a first type. (9) correspond to a sequence of a second type. (10) correspond to a sequence of a third type. The three different sequences are used for different function. In this example (8) is used as specific sequence for capturing mRNA in reverse transcription. (9) is used as an identifier different and know for each spot. (10) is used for further molecular biology reaction.

FIG. 6 2D view of the assembled array and fluidics device. A stream of droplet of a first type (25,26,27) containing at least one cell or more cells, is introduced in the fluidic chamber. The droplet of a first type contain any type of reagents suitable for phenotypic analysis. The droplets of a first type are individualized in a single compartment by buoyancy. A stream of droplets of second type are then introduced in the fluidic chamber. The droplets of second type containing reagents for molecular biology reaction are organized to have in contact a droplet of a first type. The droplet of first and second type are merged (29) applying any suitable technics. The merged droplet containing the cells, lysis agent and molecular biology reagents are put in contact to the oligo spotted at the slide surface. The oligos are then released using any type of oligo cleavage. The molecular biology reaction start upon the cell becomes lyzed in presence of molecular biology reagents and the release of spotted oligos in such example.

FIG. 7 Microfluidic workflow according to one aspect of the present invention.

FIG. 8 Example of the microfluidic device and droplets trapping in the reservoirs. The cells droplets (small droplet) are trapped in a first reservoir; the reagent droplets (bigger droplets) are trapped by two pillars to temporarily locate the 2 droplets physically where the oligonucleotides have been spotted. Fusion of the two droplets and wetting of the droplet to the oligonucleotide surface will mix the 3 reservoirs together: cell droplet, reagent droplet and oligonucleotides.

FIG. 9 Prototype of the full assembled chip. The full array consists of 6 different fluidic chambers (5) containing the spots and the cavity for trapping the droplets. The droplet are injected through the chip using a first inlet (connector) channel (3). The excess of oil or droplets leaves the chamber (5) using an outlet channel (4). The carrier oil is injected through a second inlet channel (1). The droplet fusion requires an injection of PFO10% in the chamber (5) using a third inlet channel (2). The droplets are trapped in the cavities organized in the fluidic chamber (5),In the full chip other fluidic chamber are also present and can be used independently (6, 7, 8, 9, 10).

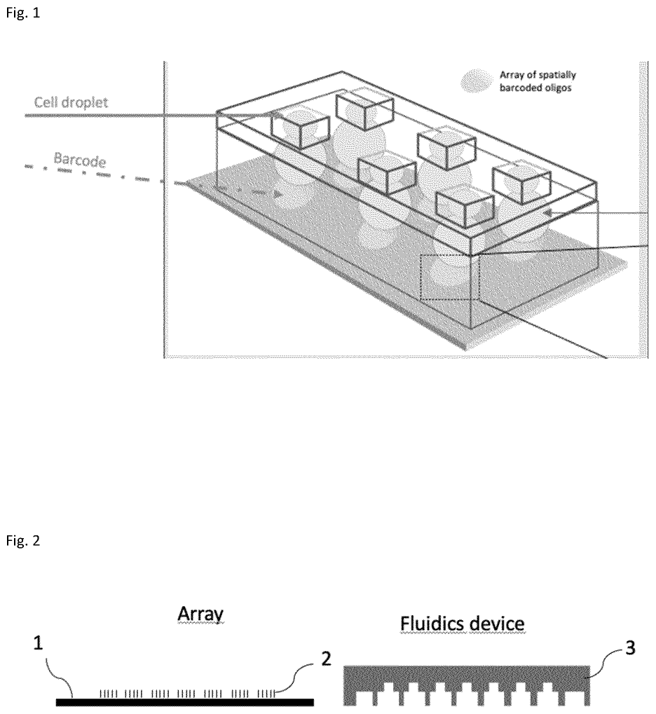

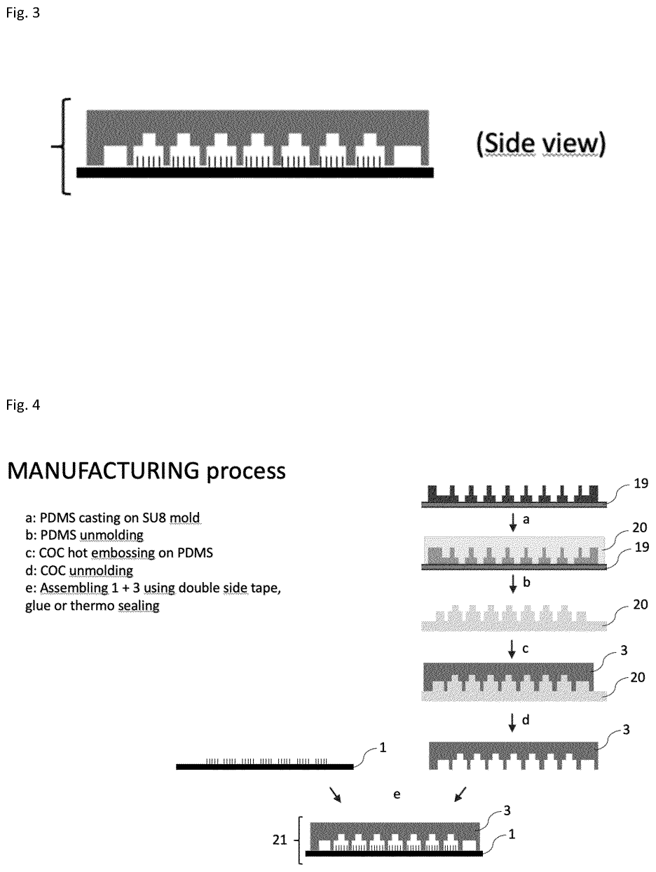

### DETAILED DESCRIPTION OF THE INVENTION

The invention relates to a microfluidic system comprising:
a) a solid support comprising at least a first group of oligonucleotides,
   i. wherein each oligonucleotide in said group comprises a nucleic acid sequence of a first type, of a second type and/or a further type,
   ii. wherein said nucleic acid sequence of a first type is a barcode sequence together in a group of oligonucleotides on said solid support,
   iv. wherein the first and further oligonucleotide groups are spatially separated on said solid support,
b) wherein said one or more groups of oligonucleotide groups on said solid support are within separate reservoirs of the microfluidics system,
c) wherein the one or more reservoirs are accessible to fluids, cells, chemicals and/or microdroplet by means of channels, and
d) wherein each reservoir comprises comprising a group of oligonucleotides on said solid support is also trap for a microfluidic droplet.

In the context of the present invention, the term “microfluidic system” refers to a device comprising at least one microfluidic channel. Said channel may be made by any method known in the art and comprising milling, etching, ablation, embossing or molding into a material (glass, silicon, ceramic paper, hydrogel or polymer such as PDMS, TPE, PS, PEGDA, PFEP/PFA/PFPE, PU, PM MA, PC, COP or COC—and composites of said materials).

The microfluidic system may also comprise a sorting system. Microfluidic cell sorting systems are known to the person skilled in the art and described, for example, by Wyatt Schields (Wyatt Schields et al. 2015, Lab Chip 15(5):1230-1249).

In the context of the present invention, the term “oligonucleotide” refers to an oligomer or polymer of either ribonucleic acid (RNA) or deoxyribonucleic acid (DNA), as well as non-naturally occurring oligonucleotides. Non-naturally occurring oligonucleotides are oligomers or polymers which contain nucleobase sequences which do not occur in nature, or species which contain functional equivalents of naturally occurring nucleobases, sugars or inter-sugar linkages.

In one embodiment, the oligonucleotides may comprise one or more nucleic acid sequences selected from the group of a first type, of a second type and/or of a third type. In one embodiment the nucleic acid sequence of a first type may be a barcode sequence. As used herein, the barcode sequence is used to identify nucleic acid molecules, where sequencing can reveal a certain barcode coupled to a nucleic acid molecule of interest. In the context of the present invention, it is sufficient that at least a portion of the barcode sequence is recognized in the sequence-specific event to identify an oligonucleotide of interest.

In the system according to the invention the barcode sequence of each group is known and the position on the solid support is known.

In the system according to the invention at least parts of the system are optically transparent and allow for optical analysis of a cell(s) trapped in said reservoir. Ideally, the transparent part is adjacent to the oligonucleotide groups.

In the system according to the invention each group of oligonucleotides comprises between 10⁴ and 10¹¹ oligonucleotides. It is preferred if a group has about 10⁹ (+/−25%).

In the system according to the invention the cell trap is a cavity of the following dimensions about 10 μm to 200 μm (+/−25%). The dimension is set to accommodate droplets containing one cell or two cells, in some embodiments more than two cells, preferably containing small cells like bacteria and bigger cells like neuronic cells.

In the context of the present invention, the term “cell” refers to any eukaryotic cell. Eukaryotic cells include without limitation epithelial cells, immune cells (such as lymphocytes, neutrophils, and monocytes/macrophages), hematopoietic cells, bone marrow cells, osteoblasts, cardiomyocytes, hepatocytes and neurons. Also, as used herein, and unless otherwise specified, the term “cell” zo refers to a “single cell”.

In the context of the present invention, the term “reservoir(s)” refers to any physical location of a materials (for example, fluids, cells, particles, droplets) such as materials are stored/located temporarily or permanently to a given position in the device. The reservoirs may or not prevent materials to flow, connect, interact, touch, communicate with each other.

In one embodiment of the present invention, it is understood that the oligonucleotide groups on the solid support are not physically in the reservoirs but should be interpreted as located on the solid support in correspondence of the reservoirs. Therefore, there are no reservoirs on said solid support comprising the oligonucleotide groups. This is also evident from the figures provided herein.

In another embodiment of the present invention, the oligonucleotide groups may be conceived physically in the reservoirs.

In the system according to the invention spatial separation of oligonucleotide groups is at least 100 nm and no more than 1,000 μm (+/−25%).

The inventor has found that this spatial separation is essential to avoid contamination between different the spotted DNA or different reservoir (droplet). Such contamination would result in a phenotype-genotype linkage misassignment or assignment to multiple droplets, thereby failing to identify the correct the phenotype/genotype linkage. Also, another parameter to be considered would be the size of the droplets. In this regard, reducing the spatial separation below the claimed range would compromise chemo-mechanical-physical event or reaction occurring in said droplet, such as the efficiency of a reverse transcription (RT) reaction.

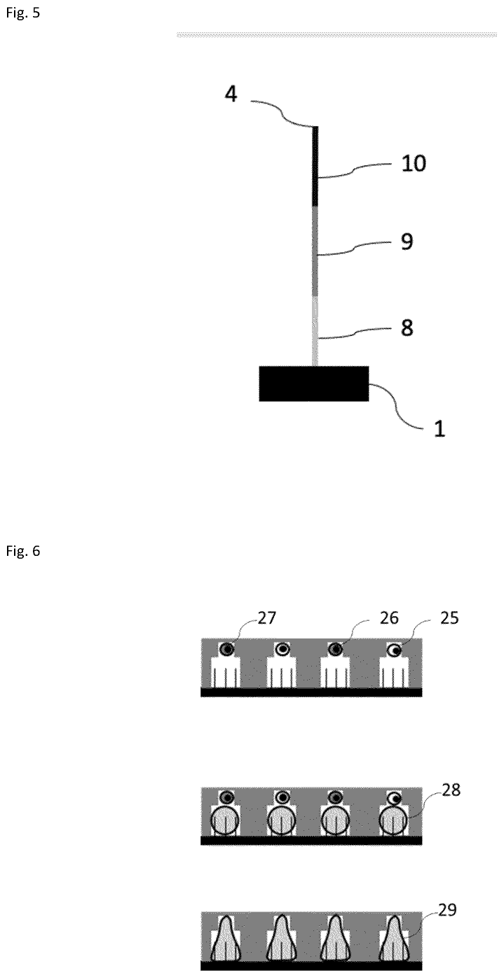

In the system according to the invention the oligonucleotides in a group comprise a nucleic acid sequence of a second type which may be a universal sequence and a further sequence type which may be a hybridizing sequence or a primer sequence and a further sequence type which by a hybridizing sequence. The oligonucleotides in each group are identical. We refer to FIG. 5. Typically, they are attached at the 5′-prime ends. Ideally, the oligonucleotide has different sequence parts which serve different purposes, such as, i) barcoding, ii) priming, or iii) hybridizing.

The invention also relates to a method of attaching an oligonucleotide to a biomolecule in a cell, the method comprising:
a) providing a microfluidic system according to the invention,
b) encapsulating a first cell in a first droplet,
c) trapping said cell droplet in said reservoir,
d) merging a second droplet comprising a lysis composition with said first droplet, thereby allowing an oligonucleotide of said solid support to attach a nucleic acid in said cell.

Strictly speaking the oligonucleotide is not attached to the cell-surface. It is attached to a nucleic acid in a cell and/or a biomolecule in a cell. The cell is brought into the vicinity of the oligonucleotides. As used herein, the expression of “attaching an oligonucleotide to a biomolecule in a cell” refers to the process of “binding” or “hybridizing” an oligonucleotide to a selected target biomolecule in a cell. As used herein the term “biomolecule” refers to any oligonucleotide, single-or double-stranded DNA or RNA. These oligonucleotides than bind a biomolecule, preferably a nucleic acid in said cell. The nucleic acid may be selected from, DNA, RNA, tRNA, mRNA, genomic DNA, ribosomal RNA, chromatin or the like. The cell may or may not be dissolved/lysed in the process of binding. In a preferred embodiment that oligonucleotides bind a nucleic stemming from the cell, the cell is lysed and the bound nucleic acids are then analyzed further.

Herein a “droplet” generally refers to a measure of volume. A “droplet” refers in context of the present invention, to an isolated portion of a first fluid that is surrounded by a second fluid. The term “droplets” used in context of the processes of the invention includes droplets of a first type, droplets of a second type, droplets of a third type, droplets of a fourth type, such as droplet comprising single cells, reagents or fused droplets, or a plurality of said droplets.

The “droplet” may have an average volume of less than 5 nL, such as less than 4 nL, less than 3 nL, preferably less than 3 nL. In some embodiments, an average volume of less than 3 nL, less than 2.5 nL, less than 2 nL, less than 1.5 nL, less than 1 nL, less than 0.5 nL, for example 0.1 nL to 3 nL, 0.5 nL to 3 nL, 1 nL to 3 nL, typically, 1 pL, 10 pL, 20 pL, 30 pL, 50 pL, 0.1 nL, 0.5 nL, 1 nL, 1.2 nL, 1.4 nL, 1.6 nL, 1.8 nL, 2.0 nL, 2.2 nL, 2.4 nL, 2.6 nL, 2.8 nL, 3 nL.

Accordingly, the “fused droplet” may have an average volume of less than 10 nL. In some embodiments, an average volume of less than 9 nL, less than 8 nL, less than 7 nL, less than 6 nL, less than 5 nL, less than 4 nL, less than 3 nL, less than 2 nL, less than 1 nL, less than 0.5 nL, for example 0.1 nL to 10 nL, 0.1 nL to 8 nL, 0.1 nL to 6 nL, 0.1 nL to 5 nL, such as 0.1 nL to 3 nL, 0.5 nL to 5 nL, 0.5 nL to 3 nL, 1 nL to 3nL, typically, 0.1 nL, 0.5 nL, 1 nL, 1.2 nL, 1.4 nL, 1.6 nL, 1.8 nL, 2.0 nL, 2.2 nL, 2.4 nL, 2.6 nL, 2.8 nL, 3 nL, 4 nL or 5 nL, such as 11 pL to 8000 pL.

After the merging of the first and the second droplet takes place, a reaction step is preferably performed which is selected from the group comprising, a cell-cell interaction, exposure to one or more substances, exposure to one or more dyes or one or more antibodies, cell lysis, nucleic acid ligation, nucleic acid amplification, nucleic acid hybridization, nucleic acid sequencing and/or a reporter or viability assay.

This is the essence of the invention. Once the single cell is located in the trap it can be analyzed. The analysis is aided by (1) the phenotypic analysis of the cell(s) using microscopy readout and (2) the spatial barcode of the oligonucleotide which is bound to the solid support and can attach to the nucleic acids of the single cells. In the context of the present invention, the term “spatial barcode” refers to a specific position of the barcode on the surface of the microfluidic chip or slide.

Preferably and additionally the phenotype of one or more cells in the one or more reservoirs is analyzed and said phenotype analysis is done
a. before merging the droplet,
b. after merging the droplets,
c. before the reaction according to claim 4, or
d. after the reaction according to claim 4.

Preferably, the barcode of the oligonucleotide attached to said solid support is used to identify a particular cell in a particular reservoir. The oligonucleotide may also be used in a reaction, such as a PCR. In this case the amplification product would comprise the barcode and sequences from the single cell. Then, the cell phenotype could be coupled to the barcode and thereby the position on the solid support.

Ideally, analyzing the phenotype comprises at least one method selected from the group of fluorescent imaging, bright field microscopy, fluorescence microscopy, confocal microscopy, time-lapse analysis, sequencing, qPCR, isothermal amplification, and e.g. RTqPCR.

The invention further relates to a method for determining a phenotype and/or a genotype of a single cell, the method comprising:
a) providing a microfluidics system comprising at least one microfluidic channel, at least a collector system comprising a plurality of reservoirs,
b) encapsulating at least one cell of a plurality of cells of a first type separately into a droplet of a first type,
optionally co-encapsulating a cell of a second type from a plurality of second type cells into each of the droplets of a first type,
c) flowing a plurality of droplets of a first type in a microfluidic channel of the microfluidics device and trapping inside each reservoir of the microfluidics device a droplet of a first type, optionally analyzing a phenotype within the droplet comprised within the reservoir,
d) flowing a plurality of droplets of a second type in a microfluidic channel and trapping inside each reservoir a second droplet of a second type,
e) merging the droplets of a first type with the droplet of a second type inside the reservoir,
f) performing at least one reaction inside the merged droplet obtained in e) and determining a readout of the reaction.

The microfluidic method for assigning a genotype to a given phenotype of interest disclosed herein presents several advantages over the methods known in the art. One advantage of the method according to the present invention is that said method enables a phenotype (including but not limited to functional read-out for an agonistic and/or antagonistic assay) assessment based on interaction, recognition, labelling, staining, imaging and/or microscopy followed by a genotype assay (including an internal messenger molecule measurement), while retaining precise phenotype/genotype relationship of each individual cell. A further advantage of the present method results in providing an improved reliability by using a two-step phenotype measurement. Lastly, the present method is characterized by a great versatility, since it can be adapted for performing different functional assays by adding a second phenotypic droplet to the first phenotypic droplet.

The aforementioned advantages are disclosed hereinafter in aspects and embodiments characterizing the present invention. Implementation of the invention is provided in examples and figures sections.

According to one aspect of the present invention, there is provided a microfluidic method for assigning a genotype to a phenotype of interest in at least one droplet, the method comprising the steps encapsulating at least one cells of a plurality of cells of a first type into a plurality of droplets of a first type, wherein each droplet of a first type comprises a single cell or no cell. Optionally cell of a second type may be co-encapsulated with a cell of a first type inside a droplet of a first type. The method according to the invention further comprises injecting and/or flowing such droplets of a first type, comprising a single cell of a first type, and optionally additionally a single cell of a second type, inside a channel of a microfluidics device. The microfluidics device further comprising at least one collector system comprising a plurality of reservoirs. The droplets of a first type may then be trapped separately inside such reservoirs. Optionally the droplets of a first type may be analyzed within the reservoirs to determine a phenotype of the cell of a first type or of the cells of a first and a second type, using, without being limited to by imaging or microscopy. Further methods for determining a phenotype according to the method of the present invention are described herein.

Subsequently, droplets of a second type, comprising reagents for performing one or more reactions are injected into and/or flowed through a channel of the microfluidics device, such that the droplets of a second type may get trapped separately inside each of the reservoirs of the microfluidics device. Consequently, each reservoir of the microfluidics device comprises one droplet of a first type and one droplet of a second type. A droplet of a first type may be fused or merged with a droplet of the second type according to a method known in the art. After fusion of said droplets one or more reactions may be initiated or take place resulting in one or more readouts or a signals, which can be detected. Such readout may be a genotyping reaction, a phenotyping reaction or a combination of both. Consequently, in one embodiment of the invention the second droplet comprises the reagents required for a genotyping and/or a phenotyping reaction.

The reservoirs of the microfluidics device may comprise on the bottom and linked to a solid support a plurality of oligonucleotides. Such oligonucleotides may be grouped into at least a first group, wherein each group is spatially separated from other groups, comprised in other reservoirs of the device. Groups of oligonucleotides comprised within the same reservoir might comprise the same nucleic acid sequence of a first type, which may be a barcode sequence. Different reservoirs of the microfluidics device might comprise the same or different barcode sequences. In one embodiment each reservoir comprises oligonucleotides with barcodes unique to said reservoir, enabling the identification of oligonucleotides and/or nucleic acids attached to said oligonucleotides comprised or located within the same specific reservoir. Therefore, the method according to the invention facilitates the linking of a specific reservoir to a specific barcode and hence to a specific phenotype of a cell detected within said reservoir. Consequently, if the genotype of a cell trapped within a specific reservoir is determined, the detected barcode sequence can be linked to a phenotype detected in a specific reservoir.

Those of ordinary skill in the art will be aware of techniques for preparing microfluidic droplets. Techniques for encapsulating cells within microfluidic droplets are described for example in Mazutis, et al. 2013, Nat. Protocol 8:870-891. In one example, droplets are prepared prior to injection in a separate microfluidic device.

In order to carrying out the method according to one aspect of the present invention, the microfluidic chip further comprises at least one collector system, comprising a plurality of reservoirs, traps or cavities. In the context of the present invention, the terms “reservoir”, “trap” and “cavity” may be used herein interchangeably. In the context of the present invention, at least one droplet moves into one reservoir of said plurality of reservoirs by buoyancy, hydrodynamic or physical forces. Preferably, said droplet collecting step is performed by buoyancy force.

Further features of the microfluidic chip for carrying out the method according to one aspect of the present invention are provided later in this section.

The method disclosed herein encompasses flowing droplets comprising single-cells of a first type, and, optionally of a second and/or of a third type.—A cell type is a classification used to identify cells based on their morphological or phenotypical features. As used herein, the term “flowing” refers to a plurality of droplets flowing inside the microfluidic chip, comprising a single cell. Said cells may be of a first type, a second type or a third type according to their cell type, certain genetic or gene expression differences, their origin or certain cellular functions.

In the method disclosed herein droplets of a first type may comprise encapsulated cells of a first type or co-encapsulated cells of a first and of a second type.

In a further embodiment the droplet does not comprise the cell but the biomolecules stemming from the cell or a fraction thereof.

Droplets of a second type may comprise reagents for carrying out, inducing, enabling or supporting a reaction or a detectable event within the merged droplet, which may be acquired by merging a droplet of a first type with a droplet of a second type.

In the context of the present invention, a cell of a first type may be a bacterial cell (for example, E. coli and B. subtilis) it can be a eukaryotic cell including, without limitation, epithelial cells, immune cells (such as lymphocytes, neutrophils, and monocytes/macrophages), hematopoietic cells, bone marrow cells, osteoblasts, cardiomyocytes, hepatocytes and neurons, like yeast (for example, Saccharomyces and Pichia), it can be an insect cell, it can be a eukaryotic or a prokaryotic cell or a virus or a pseudo particle (e.g., small molecule aggregate as DNA forming particles, DNA complexes or DNA aggregates). There are no limitations here. Preferred cells include immune cells such as B-cells, T-cells, NK-cells, NKT cells, macrophages, or dendritic cells.

In the context of the present invention, a phenotype of interest may be presence of surface marker, changes in composition of surface markers, activation or blockade activity, intracellular modification, production of molecules such as metabolites, peptides, proteins, cell behavior such as cell viability, cell interaction, cell displacement.

In the context of the present invention, a genotype of interest may be transcripts mRNA, tRNA, siRNA, miRNA, piRNA, DNA such as genomic, mitochondrial DNA, epigenomics such as modified DNA, chromatin structure, modified RNA, or structural organization of the molecule thereof.

In another embodiment, a cell of a first type may be a reporter cell. Differently, a cell of a second type may be a secreting cell, preferably an antibody secreting cell, wherein said antibody is against a membrane target presented by said reporter cell. Therefore, in the context of the present invention, a cell of a first or a second type may possess a first phenotype. Similarly, a cell of a third type may possess a second phenotype.

According to another embodiment of one aspect of the present invention, the cells of a first type may be an antibody secreting cell and the cell of second type may be a reporter cell. As used herein, the term “reporter cell” refers to a cell comprising a reporter gene, or protein or lipid, or chemical compound that will ultimately refer to the functional effect of said agent acting on the reporter system.

According to another embodiment of one aspect of the present invention, the cells of first type may be a T-cell and the cell of second type may be an antigen presenting cell.

As used herein, the term “reporter cell” refers to a cell comprising a reporter gene, a protein, a lipid, or a chemical compound which, when expressed, produces a reporter signal that is readily measurable, e.g., by biological assay, immunoassay, radioimmunoassay, or by colorimetric, fluorogenic, chemiluminescent method.

According to one embodiment of one aspect of the present invention, the single cell comprising, or co-encapsulated cell droplets has a volume ranging from 10 pL to 10 nL.

In an embodiment of the method according to the present invention, each cell of a first type comprised in a droplet may be discriminated from another cell of a second type comprised in a droplet by using a label system, such as Calcein AM for secreting cells and CellTracker Red for reporter cells. A further selection measure may be represented by using a secondary, fluorescently labelled detection reagent, an AlexaFluor647 labelled, Fc-specific anti-IgG F(ab′)2 (red fluorescence), or an indirect detection (reagent coupled with for example biotin, with streptavidin) to visualize binding of an immunoglobulin on the target on the reporter cell.

In one embodiment of the invention complex analysis of cell-cell interactions, for instance, antigen presenting cells co-encapsulated with T-cells or plasmablast cells secreting antibodies against membrane presented targets on cells, can be performed in a high throughput manner.

Importantly, cellular assays can be performed in droplets to measure functional responses induced by a compound, including, but not limited to, calcium flux, cyclic AMP, beta-arrestin recruitment, internalization, cytokine secretion, chemokine secretion, receptor dimerization, actin polymerization, cell division, cell cycle blocking or phosphorylation, MAP kinase activation, apoptosis, necrosis, granules, di-multimerization assay over expression and presentation of specific molecules at the surface of the cell and/or internally.

Secreting cells and reporter cells may be co-encapsulated and the number of said co-encapsulated cell can be estimated using the Poisson distribution. In the context of the present invention, the co-encapsulation process is performed by increasing the lambda value above 0.5 for the Poisson distribution of reporter cells to achieve co-encapsulation rates of above 50% of secreting cell and reporter cell into droplets. Alternatively, specifically engineered devices can be used to achieve the same result or higher performance.

In the context of the present invention, the encapsulation or co-encapsulation of cells of a first or of a first and second type in droplets of a first type may be carried out within the same chip in which the analysis is performed or off-chip, or within another chip or microfluidics device. Off-chip may refer to a separated area outside the microfluidic chip. As a consequence, in one embodiment a plurality of droplets can be stored off-chip, for example, in test tubes and manipulated or analyzed by reinjecting said plurality of droplets into the microfluidic chip.

The method according to the present invention may include at least one incubation step, which can be timed to allow the occurrence of a first or a second detectable event or reaction.

As used herein, the term “detectable event”, “detectable reaction” or “reaction” refers to any chemo-mechanical-physical event or reaction that may be observed and/or detected. Depending on the phenotypic assay, at least one single cell may be assayed for selected parameters using any suitable assay method, which may be qualitative and/or quantitative. Suitable detection methods may include spectroscopic methods, electrical methods, hydrodynamic methods, imaging methods, microscopic methods, reporter assays, methods for detecting emitted light or fluorescence and/or biological methods. The terms “detectable event”, “detectable reaction”, “reaction” or “assay” may be used herein interchangeably.

A reaction or chemo-mechanical-physical event may be the staining of a cell or the absence of a staining with a dye or any other reagent known to the skilled person, an amplification reaction, a real-time or qPCR reaction, a reverse transcription reaction, a ligation, a viability assay or toxicity assay, a sequencing reaction, the detection of and/or the binding of an antibody with an antigen, a fluorescence reaction or reporter assay, a killing assay, the secretion of molecule, the cell-cell interaction, the exchange of material from cell to cell, a change of morphology reaction, a measurement of viscosity and/or aggregation, the synthesis of a product of molecule, emission of fluorescence or the like.

As reported above, one of the advantages of the method disclosed herein lies in its versatility. Therefore, a further stream of droplets of a third type comprising reagents used for a second reaction or reaction step may be also injected into the microfluidic chip to contact at least one droplet comprising at least one cell and collected within a cavity or reservoir of the collector system to generate at least one fused droplet comprising a first phenotype and a second phenotype.

According to another embodiment of one aspect of the present invention, the single-cell droplet of third type has a volume ranging from 10 pL to 10 nL, preferably 50 pL to 1 nL.

According to one embodiment of one aspect of the present invention, the fused droplet has a volume ranging from 20 pL to 10 nL, preferably 50 pL to 1 nL.

According to another embodiment of one aspect of the present invention, the single-cell droplet of a second type or third type may comprise one or more dyes for staining cells, reagents for sequencing reactions comprising a fluorescent substrate, reverse transcription reagents, a lysis buffer, PCR or qPCR reagents, reagents for a reporter and/or viability assay and/or reagents for detecting the binding of an antibody, or the like.

A sequencing and/or reverse transcriptase reaction may analyze genes representing the whole genome or transcriptome of lysed cells, or a panel of RNA or DNA used as an indicator of effector function, or a random set of RNA or DNA, or epigenetic information (protein, DNA, RNA and structural configuration), a combination of RNA and DNA, a protein from said cells or from said compartment.

A droplet of a first type comprising a cell with a first phenotype and, optionally, also a co-encapsulated cell with a second phenotype, collected or trapped within a reservoir, may be optionally imaged, and may be subsequently contacted by a stream of droplets of a second type comprising reagents for performing genotyping reactions, thereby facilitating a droplet of a second type being trapped inside said reservoir and subsequently being merged to said droplet of a first type. After the merging of the droplet of a first type with a droplet of a second type a genotyping action can take place.

According to one embodiment of the present invention, the droplet of second type may comprise reagents for at least a first reaction. In another embodiment the droplet of a second type may comprise reagents for a first and a second reaction. In another embodiment the droplet of a second type may comprise reagents for a first, a second and at least a third reaction. The first, second and any further reactions may be performed in a consecutive order or in parallel inside the merged droplet.

According to one embodiment of the present invention, a droplet of third type may comprise reagents for at least a second reaction. Said droplet of a third type may be flowed through a microfluidic channel to a reservoir comprising the merged droplet, which was obtained by merging a droplet of a first with a droplet of a second type, both trapped inside the same reservoir. Said droplet of a third type may subsequently be trapped inside said reservoir comprising said merged droplet after the occurrence of a first and/or a second reaction inside said merged droplet. Said droplet of a third type may be merged with said “merged droplet” inside said reservoir and a second and/or a third reaction may take place.

According to another embodiment of the present invention, the droplet of second type may comprise reagents for chromatin digestion (including but not limiting to MNAse, DNAse, Tagmantase) and the droplet of the third type may comprise reagents for sequencing reactions comprising ligase (or transposase) and buffers reagents, such that, when said droplet contacts a surface or solid support spotted with barcoded DNA, the capture of chromatin fragments of interest is enabled. These chromatin fragment may represent mono, di, tri or array of nucleosomes; they may represent digested DNA, of a length from 10 bp to several Mb.

In another embodiment of the present invention, a phenotype of interest may comprise the production of an antibody having effector function (binding, cross reactivity, specificity, agonist, antagonist, allosteric modulator), including activation/inhibition of downstream signaling cascades from reporter cells; production of a cytokine and/or granules (e.g., perforin, granzyme) and/or induction of expression of cell surface markers (e.g., CD69, CD137, CD4OL, OX40, PD1) induced by the TCR-MHC peptide complex from T cells and APC respectively; it may comprise activation/inhibition of cell metabolism (e.g., production interleukin, cytokine, chemokine; apoptosis, or necrosis).

Reagents for performing a genotyping reaction are known to a skilled person. Generally, said reagents may comprise, without being limited to, fluorescent substrates, reverse transcription reagents and lysis buffer and any source of barcoded libraries, oligonucleotides, primers, barcodes, polymerases, ligase, transposase, and amplification reagents. As used herein, the term “genotyping” refers to the process of determining the nucleic acid sequence of a single cell by using biochemical methods and/or determining structural features of cellular genome/transcriptome.

Methods for fusing droplets are also known in the art as described, for example, by Mazutis (Mazutis et al. 2012, Lab Chip 12, 1800-1806). Said methods may comprise the addition of surfactants such as perfluoro-octanol, providing special microfluidic channel geometries and/or application of electric fields or acoustic waves. In the context of the present invention, the fusing step is preferably performed by applying an electrical field. The fusing step may be performed in predefined areas of the chip to selectively fuse the droplets contacting said predefined area. The terms “fusion” and “merging” may be used interchangeably herein.

In the context of the present invention, droplet fusion is achieved by applying of electrical fields with a frequency ranging from 2 kHz to 40 kHz and a voltage ranging from 500 V to 20000 V for the time necessary for achieving a fusion efficiency of 80% to 100% between the two droplets involved in said event. Higher and lower frequency and voltage may be applicable as well depending on, for example, to the surface tension between the droplets, surfactant concentration, droplet volume ( . . . ) to be fused.

According to other embodiments the fusion is performed but not limited to laser/light induced, chemical and acoustic fusion. According to one embodiment of one aspect of the present invention, the fusing step (i) is performed by means of an electrical arrangement comprising a plurality of electrodes. In the context of the present invention, said plurality of electrodes are preferably made onto the glass array chip in a row and column format from indium tin oxide with a thickness of 300-600 Å. The electrodes may be structured by photolithography and indium tin oxide sputtered onto the glass chip. According to a further embodiment of one aspect of the present invention, the fusing step (i) is performed by means of an electrical arrangement comprising a plurality of electrodes arranged on the top side of the microfluidic system in a row format and on the bottom side in a column format, or vice versa. An exemplary device for generating focused electric field may be an anti-static gun.

The inventors have found that by activating a defined combination of row and column indices, it is possible to selectively fuse droplets. This procedure is particularly advantageous because it provides an additional selection step in the screening process.

Also, the selective fusion of droplets is used to release and/or render accessible the second or third droplet content to the first droplet, potentially having phenotype of interest and for which genotypic information is desired. Further, the selection of functional antibodies for further processing, for example subsequent sequencing and cloning, expression and validation, increases the probability of obtaining bona fide hits with desired properties for secondary screens.

Following droplet fusion, a variety of one or more reactions may be initiated and/or performed in said droplet, such as, but not limited to, fluorescence staining of a cell or a component of a cell, sequencing or sequence capture reactions, amplification or ligation reactions, reporter assays.

Detection of a first and/or a second detectable event according to the present invention may include the use of stains, dyes, labels, enzymes, substrates, cofactors, and/or specific binding partners (SBPs). According to the phenotype of interest intended to be detected the skilled person knows which method may be suitable. In the contest of the present invention, the detection of a second detectable event is preferably carried out by using a spectroscopic method leading to mapping, for each reservoir, the phenotype of interest comprised in at least one fused droplet located in at least one reservoir.

According to another embodiment of one aspect of the present invention, the fusing step (i) is controlled by means of electrowetting.

As used herein, the term “electrowetting” refers to the use of an electric field to alter the wetting propriety of a droplet relative to the chip surface in order to control the movement and/or shape of said droplet. In the context of the present invention, electrowetting may be used to control spreading of the fused droplet on the chip surface, without the need to utilize pumps, valves, channels and/or other similar fluid handling mechanisms. Examples of electrowetting can be found in, e.g., Pollack et al. 2000, Applied Physics Letters, 77, 1725 (describing a microactuator for rapid manipulation of discrete microdroplets that achieved transferring droplets (0.7-1.0 μl) of 100 mM KCl solution between adjacent electrodes at voltages of 40-80 V and repeatable transport of droplets at electrode switching rates of up to 20 Hz and average velocities of 30 mm/s); Fouillet et al., Proceedings of ASME ICNMM2006 4 International Conference on Nanochannels, Microchannels and Minichannels Jun. 19-21, 2006, Limerick, Ireland; Paper No. ICNMM2006-96020 (describing the use of Electro Wetting On Dielectric (EWOD) on real time PCR (Polymerase Chain Reaction) within a 64 nl microfluidic droplet).

It is important to control the behavior of the fused droplets by means of electrowetting because it may allow the incorporation within the droplet of a barcode nucleotide sequence spotted on the surface of the microfluidic chip. The droplet enters in hydrophilic contact with the slide comprising the spotted DNA. The content of the droplet is then in contact with the spotted DNA and can trigger reaction.

In some embodiment, the merged droplet contains enzymes specific capable of cleaving specific DNA sites included in the spotted DNA. This reaction is used to release the bardcoded DNA in the fused droplet.

In one aspect, the present invention provides a microfluidic chip or device comprising: two inlets and one outlet, 2,000 spatial barcodes (up to 200 k) and the corresponding reservoirs, possibly including droplet makers designs and nozzles integrated into the device.

In the context of the present invention, the microfluidic chip or device may comprise different inlets and outlets as well as different combinations of inlets and outlets. Therefore, the microfluidic chip or device may comprise at least one inlet and one outlet.

As used herein, the term “correspondence” refers to a determined position on the chip surface of the spots comprising barcodes. In the context of the present invention, said position is preferably defined on the area of the chip surface opposite to the reservoir.

According to another embodiment, each spot comprises an oligonucleotide density above of 10⁵.

According to another embodiment, each spot has a diameter ranging from 10 to 200 μm, preferably ranging from 50 to 150 μm, more preferably ranging from 60 to 80 μm.

As used herein, the term “spot” refers to a defined area of the first and/or second surface of the microfluidic chip wherein a second droplet contacts a first droplet and a coalescence/fusion event is triggered by activating a plurality of electrodes arranged on a first surface of the microfluidic chip and/or on said second surface, by means of controlling physical or chemical parameters of the fluid, e.g., temperature or ionic force.

In one embodiment, at least one droplet of the first type is fused with at least one droplet of the second type using an electrical field. In another embodiment, the fusion step results in a fusion efficiency of 80% to 100% between the droplets of the first type and the droplets of the second type, preferably 90% to 100%.

The microfluidic chip disclosed herein provides the advantage of compartmentalizing reactions in separate and distinct areas of the microfluidic chip by coalescence of selected microfluidic droplets. Therefore, the microfluidic chip according to the present invention provides an improved control of biological essays, which may occur simultaneously in different area of the chip.

Polydimethylsiloxane (PDMS) is a two-part polymer comprising a base elastomer and a curing agent. The standard mixing ratio for PDMS is 10-parts base elastomer and 1-part curing agent. In one embodiment, the first polymer solution comprises an elastomer and a curing agent in a ratio 5:1. The inventors have found that this ratio provides the desired mechanical properties for the mold.

Once the droplets are fused by using of an electrical arrangement according to the present invention, oligonucleotides may be cleaved from the chip surface by any suitable method. Preferably, the oligonucleotides are cleaved by photo-cleavage.

In one embodiment the barcode sequence may be unique to one or more reservoirs of the microfluidic device and therefore facilitate the identification of single cells trapped and analyzed withing respective reservoirs. By customizing and specifically selecting the barcodes spotted in a certain location on the solid support of the microfluidics device the present method facilitates the identification and linkage of specific phenotypes detected in said specific location with the genetic information acquired by the analysis methods described herein. Therefore, the phenotype of a single cell, which is trapped within a specific reservoir of the microfluidics device, can be linked to the genotype of said single cell.

The term “nucleic acid” as herein used generally refers to at least one molecule or strand of DNA, RNA, miRNA or a derivative or mimic thereof, comprising at least one nucleobase, such as, for example, a naturally occurring purine or pyrimidine base found in DNA or RNA. The term “nucleic acid” encompasses the term “oligonucleotide”. A nucleic acid herein may also be attached to one or more proteins.

“RNA” herein refers to, but is not limited to, functional RNA, such as mRNA, tRNA, rRNA, catalytic RNA, siRNA, miRNA, piRNA, ncRNA, IncRNA . . . and antisense RNA. In one preferred embodiment, RNA refers to mRNA.

The term “oligonucleotide” refers to at least one molecule of about 3 to about 500 nucleobases in length. For example, the oligonucleotide may have a length of at least 3 nucleobases, at least 10 nucleobases, at least 30 nucleobases, at least 50 nucleobases, at least 100 nucleobases. In some cases, the oligonucleotide may have a length of no more than 100 nucleobases, no more than 50 nucleobases, etc. Combinations of any of these are also possible, e.g., the length of the oligonucleotide may be between 3 and 300 nucleobases, preferably 3 and 200 nucleobases, more preferably 3 to 100 nucleobases.

When performed in droplets, the method according to the invention further comprises the step of recovering or collecting the fused droplets at the outlet of the channel after the reaction steps performed inside the reservoirs.

According to another aspect, disclosed herein is a method of manufacturing a microfluidic system according to the present invention, comprising the steps of
a. generation of mask comprising the design of the fluidic device,
b. photoactivation of resin, preferably SU8, for positive replication of the negative design printed in the mask,
c. excess resin removal using appropriate solvent for non-photo activated resin,
d. polymer casting (PDMS) the microfluidic system on the resin, preferably SU8 mold,
e. polymer reaction for solidifying, typically PDMS polymerization,
f. unmolding the casted and solidified polymer,
g. COC hot embossed on solidified polymer (PDMS),
h. COC unmolding,
i. assembling of the array including oligos and the COC fluidic part preferably using thermo-sealing, double side tape or any other sealing technic.

### EXAMPLE

Capture the cell sequences from a model two cell lines Jurkat (T cell type) and Ramos (B cells). To encapsulate and sort Jurkat and Ramos cells, reverse transcription (RT) has been performed in an assemble of microfluidic chamber and pre-spotted slide provided by Arbor bioscience as described in patent application WO 2018167218 A1.

### Protocol

### 1 Preparation of the Cells

Harvesting Jurkat and Ramos cells, washing 2× in 1 mL 1×PBS with 300 g spin for 6 minutes, then resuspending cells in 500 μL 1×PBS;
Labeling Jurkat cells with CellTrace FarRed (0.5 μL);
Labeling Ramos cells with CellTrace Far Red+Yellow (0.25 μL+0.25 μL);
Incubating 30 min at RT protected from light;
Adding RPMI media with 10% HI-FBS, spinning and washing with 1×PBS 2 times;
Resuspending Jurkat cells in 30 μL and Ramos cells in 200 μL of PBS;
Counting cells:
Jurkat: 4 mLn/mL
Ramos: 70 mLn/mL
Preparing cell mix (lambda=1):

| Cell mix | | | | Final |
|---|---|---|---|---|
| Reagent | Concentration | | Volume (μL) | concentration |
| Jurkat cells | 4 | mLn/mL | 15 | ~500k/mL |
| Ramos cells | 70 | mLn/mL | 1 | ~500k/mL |
| Sulphorhodamine | 250 | μM | 1 | 2.5 μM |

| | | | |
|---|---|---|---|
| Optiprep | 100% | 15 | 15% |
| 10X PBS | 10X | 8.4 | 1X |
| Water | | 59.6 | |
| Total | | 100 | |

Encapsulating and sorting cells with the integrated droplet generator+sorter with parameters:
   Aqueous phase: 50 μL/hr, Oil1: 500 μL/hr, Oil2 (Spacer): 600 μL/hr.
   Sort parameters: Sorting based on Red channel, 6000 Hz amplitude, 300V, 200 μs delay, 2 ms sort time;
Once ^{˜}30 k droplets are sorted, connecting the collection outlet to the chamber and plugging the waste channel with an Eppendorf tube, stopping the aqueous phase. Inverting the chamber and collecting droplets to rise up in the tubing until they reach the inside of the chamber, then placing the chamber on the microscope stage to observe the loading;
Decreasing the oil flow rate to ^{˜}300 μL/hr;
Once most droplet traps are occupied, flushing the remaining droplets and clamp outlet, then bringing the chamber to imaging station and imaging with brightfield, TRITC and Cy5 channels: Jurkat cells—Red only, Ramos—Yellow (lower in Red, but still detectable in Red).

### 2 RT Mix:

| | Initial | | Volume | Final | |
|---|---|---|---|---|---|
| Reagent | concentration | | (μL) | concentration | |
| 5X SSIV buffer | 5X | | 10 | 1X | |
| dNTPs | 10 | mM | 2.5 | 0.5 | mM |
| DTT | 100 | mM | 2.5 | 5 | mM |
| Igepal CA-630 | 10% | | 1.5 | 0.3% | |
| Suphorhodamine | 250 | μM | 1 | 5 | μM |
| SSIV | 200 | U/μL | 5 | 20 | U/μL |
| BmrI | 5000 | U/mL | 3 | 0.3 | U/μL |
| RnaseIN | 20 | U/μL | 2 | 0.8 | U/μL |
| Water | | | 22.5 | | |
| Total | | | 50 | | |

Encapsulating with 200 μL/hr+600 μL/hr aqueous and oil flow rates until the droplets reach end of the outlet;
Connecting the chip outlet to the fluidic chamber (the assembled chip according to the present invention) and increasing the oil flow rate to 1500 μL/hr (stop the aqueous flow), until droplets reach the middle of the chip;
Decreasing the oil flow rate to 200 μL/hr to wash away unnecessary droplets.
Once no extra droplets are in the chamber, fusing the droplets with antistatic gun, triggering for 1 minute, then fusing the droplets to the surface with 10% PFO at 200 μL/hr.
Pre-fusion:
Clamping tube with 1.5 mL Eppendorf tubes and transferring it to thermal incubator (with plate adaptor);
Running a incubation program: 10 min at 37° C., 1.5 hour at 52° C., lhour at 4° C.;
Eluting cDNA from the chamber by infusing 100 μL TE buffer, 100 μL 10% PFO and 100 μL TE buffer, then AMPure 1.0× into 20 μL water.

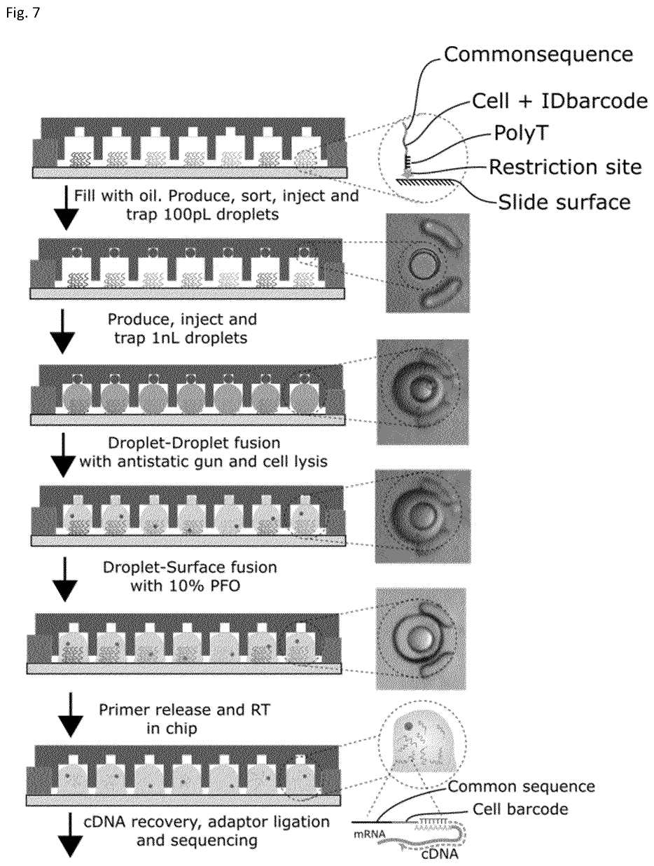

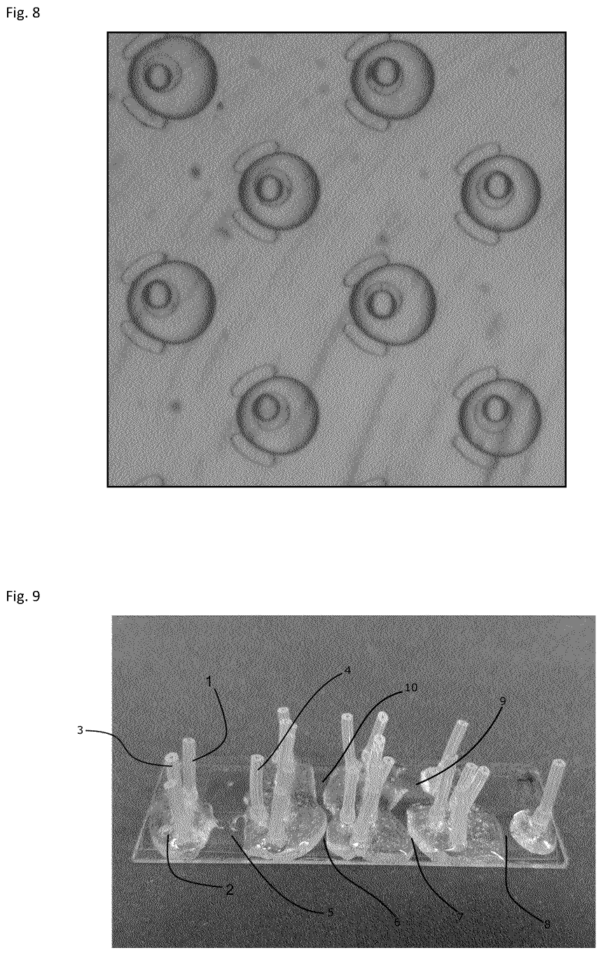

The above steps are also depicted in FIG. 7.

1. A microfluidic system comprising:
v. a solid support comprising at least a first group of oligonucleotides,
i. wherein each oligonucleotide in said group comprises a nucleic acid sequence of a first type, of a second type and/or a further type,
ii. wherein said nucleic acid sequence of a first type is a barcode sequence
iii. and oligonucleotides comprising the same barcode sequence are grouped together in a group of oligonucleotides on said solid support,
iv. wherein the first and further oligonucleotide groups are spatially separated on said solid support,
vi. wherein said one or more groups of oligonucleotide groups on said solid support are within separate reservoirs of the microfluidics system,
vii. wherein the one or more reservoirs are accessible to fluids, cells, chemicals and/or microdroplet by means of channels, and
viii. wherein each reservoir comprises comprising a group of oligonucleotides on said solid support is also trap for a microfluidic droplet.

2. System according to claim 1, wherein the barcode sequence of each group is known and the position on the solid support is known.

3. System according to claim 1 or 2, wherein at least parts of the system is optically transparent and allows for optical analysis of a cell trapped in said reservoir.

4. System according to claims 1 to 3, wherein each group of oligonucleotides comprises between 10⁴ and 10¹¹ molecules of oligonucleotides.

5. System according to claims 1 to 4, wherein the cell trap is a cavity of the following dimensions 10 and 100 μm.

6. System according to claims 1 to 5, wherein each spatial separation of oligonucleotide groups is at least 100 nm and no more than 1,000 μm.

7. System according to claims 1 to 6, wherein the oligonucleotide in said group comprises a nucleic acid sequence of a second type which may be a universal sequence and a further sequence type which by a hybridizing sequence.

8. A method of attaching an oligonucleotide to a cell, a biomolecule of said cell, or preferably a nucleic acid contained in said cell, the method comprising:
a) providing a microfluidic system according to any of the claims 1 to 7,
b) encapsulating a first cell in a first droplet,
c) trapping said cell in said reservoir,
d) merging a second droplet comprising a lysis composition with said first droplet, thereby allowing an oligonucleotide of said solid support to attach a nucleic acid in said cell.

9. Method according to claim 8, wherein after the merging of the first and the second droplet takes place, a reaction step is performed which is selected from the group comprising, a cell-cell interaction, exposure to one or more substances, exposure to one or more dyes or one or more antibodies, cell lysis, nucleic acid ligation, nucleic acid amplification, nucleic acid hybridization, nucleic acid sequencing and/or a reporter or viability assay.

10. The method of claim 8, wherein additionally the phenotype of one or more cells in the one or more reservoirs is analyzed and said phenotype analysis is done
a. before merging the droplet,
b.after merging the droplets,
c. before the reaction according to claim 9, or
d. after the reaction according to claim 9.

11. Method of claims 8 to 10, wherein the barcode of the oligonucleotide attached to said solid support is used to identify a particular cell in a particular reservoir.

12. Method according to claims 8 to 11, wherein said oligonucleotide attached to said solid support is used in a reaction step according to claim 9.

13. The method of claim 10, wherein analyzing the phenotype comprises at least one method selected from the group of fluorescent imaging, bright field microscopy, fluorescence microscopy, confocal microscopy, sequencing, qPCR.

14. Kit comprising a microfluidic system according to 1 to 7 and optionally instructions for performing the method of claims 8 to 13.

15. Method of manufacturing a microfluidic system according to claims 1 to 7, comprising the steps of
a. generation of mask comprising the design of the fluidic device,
b. photoactivation of resin, preferably SU8, for positive replication of the negative design printed in the mask,
c. excess resin removal using appropriate solvent for non-photo activated resin,
d. polymer casting (PDMS) the microfluidic system on the resin, preferably SU8 mold,
e. polymer reaction for solidifying, typically PDMS polymerization,
f. unmolding the casted and solidified polymer,
g. COC hot embossed on solidified polymer (PDMS),
h. COC unmolding,
i. assembling of the array including oligos and the COC fluidic part preferably using thermo-sealing, double side tape or any other sealing technic.
